(19) United States
(12) Patent Application Publication
(10) Pub. No.: US 2021/0260250 A1
(43) Pub. Date: Aug. 26, 2021

(54) Methods for Enhanced Endothelialization of Implanted Material

(71) Applicants: Jeffrey Scott SOBLE, Chicago, IL (US); Uri GALILI, Chicago, IL (US); Gary SCHAER, chicago, IL (US); Rush University Medical Center, Chicago, IL (US)

(72) Inventors: Jeffrey Scott SOBLE, Chicago, IL (US); Uri GALILI, Chicago, IL (US); Gary SCHAER, Chicago, IL (US)

(73) Assignee: Rush University Medical Center, Chicago, IL (US)

(21) Appl. No.: 16/973,809

(22) PCT Filed: Jun. 12, 2019
(86) PCT No.: PCT/US2019/036765

§ 371 Date: Dec. 10, 2020

Related U.S. Application Data
(60) Provisional application No. 62/684,042, filed on Jun. 12, 2018.

(51) Int. Cl. A61L 27/50; A61L 27/36; A61F 2/00; A61L 27/18

(52) U.S. Cl. CPC A61L 27/507; A61L 27/3687; A61F 2/0077; A61F 2002/0086; A61L 2430/20; A61L 2430/22; A61L 2400/18; A61L 27/18

(57) ABSTRACT

Methods and devices for increasing endothelialization on a surface of a medical device are provided. The methods include delivering the medical device to an intravascular site and the medical device has an α-gal epitope attached to the surface of the medical device.

### RELATED APPLICATIONS

The present patent application claims the benefit of the filing date of U.S. Provisional Patent Application No. 62/684,042, filed Jun. 12, 2018, the contents of which is hereby incorporated by reference.

### TECHNICAL FIELD

The present disclosure relates to the fields of cardiology and vascular surgery, and in particular to methods and devices for enhanced endothelialization of the devices.

### BACKGROUND

Prosthetic synthetic grafts such as poly(ethylene terephthalate) (PET also called DACRON) and poly(tetrafluoroethylene) (ePTFE also called GORE-TEX) are widely used in cardiac and vascular surgery since the mid-1970s. When implanting any vascular prosthetic grafts, one important goal to ensure long-term patency is achieving complete endothelialization of the luminal surface. Complete endothelialization of synthetic vascular or cardiac grafts is not achieved in many patients even decades after grafting, resulting in large luminal areas of prosthetic vascular grafts remaining without an endothelium (Marois et al., ASAIO J.45: 272.1999; Zilla et al., Biomaterials, 28: 5009, 2007). In the absence of endothelial surfacing of the graft, there is a significant increase in the risk of thrombosis. This risk factor of thrombosis, caused by the contact of non-endothelialized devices with the blood exists also with a growing number of medical devices meant to remain in contact with the bloodstream within the vascular system. These implanted medical devices include, but are not necessarily limited to: Prosthetic heart valves (mechanical or biological); Stents, Intravascular occlusion devices such as, but not limited to left atrial appendage and inferior vena cava filters. In general, the risk of thrombosis is greatest early after device/material deployment, and decreases over time due to ingrowth of an endothelial layer that separates and protects the device and synthetic grafts from the thrombogenic elements in the bloodstream. Endothelial cells express antiplatelet and anticoagulant agents that prevent platelet aggregation and fibrin formation respectively, including nitric oxide, prostacyclin 12, thrombomodulin, tissue factor pathway inhibitor (TFPI), and tissue plasminogen activator (t-PA) (Agyare and Kandimalla, J Biomol Res Ther, 3: S101, 2014; Kazmi et al., Semin Thromb Hemost, 41: 549,2015).

In order to prevent thrombus formation on intravascular devices and synthetic grafts, potent anti-platelet and anticoagulant medications are often given alone or in combination, for variable period of time. All of these medications carry a risk of bleeding, and not all patients are good candidates for these medications due to prior bleeding events or bleeding risk. Therefore, a mechanism to enhance the ingrowth of endothelium over the surface of any intravascular or intracardial device or synthetic graft would be of tremendous clinical benefit if it would reduce the risk of thrombus formation and/or the need or duration of antiplatelet therapy or anticoagulation. The present disclosure teaches the induction of endothelial growth enhancement on the surface of implanted materials such as vascular and cardiac grafts and devices by α-gal epitopes attached to the surface of such synthetic grafts and devices.

### BRIEF SUMMARY

Methods of increasing endothelialization on a surface of a medical device are provided. The methods include delivering the medical device to an intravascular site. The delivered medical device has an α-gal epitope attached to the surface of the medical device.

Implant devices are also provided. The implant devices include a medical device having an α-gal epitope attached to a surface of the medical device.

Methods of making an implant device are also provided. The methods include attaching an α-gal epitope to a surface of a medical device.

### BRIEF DESCRIPTION OF THE DRAWINGS

FIG. 1 is a schematic illustration of the enhanced endothelialization of synthetic vascular grafts and devices by anti-Gal/α-gal epitope interaction, using the non-limiting example of PET coated with α-gal epitopes (α-gal PET) as a synthetic vascular graft or synthetic blood vessel. The coating with α-gal epitopes (marked by broken line rectangles) is achieved by the use of a mixture of albumin and α-gal albumin (albumin carrying synthetic α-gal epitopes linked to albumin by as spacer). The diagonal short lines between the albumin boxes represent cross-linking glutaraldehyde molecules. Upon reperfusion of a blood vessel that has an α-gal PET graft, the natural anti-Gal antibody binds to the α-gal epitopes coating the luminal surface of the synthetic graft. Monocytes/macrophages in the blood attach via their Fcγ receptors (FcγR) to the Fe portion of anti-Gal IgG molecules attached to the α-gal epitopes. The attached cells are induced to polarize into M2 macrophages that secrete vascular endothelial growth factor (VEGF). The cytokine VEGF diffuses in between the PET fibers and subsequently slowly leaches out of the α-gal PET. The released VEGF attracts endothelial cells and progenitor endothelial cells to adhere to the surface of the synthetic graft, proliferate and cover the surface with an endothelial cell lining that decreases thrombosis at the area of the synthetic graft.

FIG. 2 is an illustration of the steps of the endothelization processes occurring when an α-gal PET medical graft is implanted.

### DETAILED DESCRIPTION

For the purpose of promoting an understanding of the principles of the invention, reference will now be made to embodiments, some of which are illustrated in the drawings, and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. In the discussions that follow, a number of potential features or selections of assay methods, methods of analysis, or other aspects, are disclosed. It is to be understood that each such disclosed feature or features can be combined with the generalized features discussed, to form a disclosed embodiment of the present invention.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention.

The uses of the terms “a” and “an” and “the” and similar references in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”) provided herein, is intended merely to better illuminate the invention and does not pose a limitation on the scope of the invention unless otherwise claimed. No language in the specification should be construed as indicating any non-claimed element as essential to the practice of the invention.

The term “α-gal epitope” or “α-gal” as used herein, refers to any molecule, or part of a molecule, with a terminal structure comprising Galα1-3Galβ1-4GlcNAc-R, Galα1-3Galβ1-3GlcNAc-R, or any carbohydrate chain with terminal Galα1-3Gal at the non-reducing end, or any molecule with terminal α-galactosyl unit capable of binding the anti-Gal antibody.

As used herein, intravascular refers to a component that is positioned, in whole or in part, in a patient's vasculature, within a patient's heart, or within both.

As used herein, the term “medical devices” includes any material or device that is used on, in, or through a subject's body in the course of medical treatment for a disease or injury. Medical devices include manufactured and biologically sourced materials and devices.

The term “therapeutic effect” or “treatment” as used herein means an effect which induces, ameliorates or otherwise causes an improvement in the pathological symptoms, disease progression or physiological conditions associated with a disorder, for example thrombosis, in a human or veterinary subject. The term “therapeutically effective amount” as used with respect to an agent means an amount of the agent which imparts a therapeutic effect to the human or veterinary subject.

The term, “VEGF” as used herein, is an abbreviation for vascular endothelial growth factor.

Methods of Enhancing Endothelialization

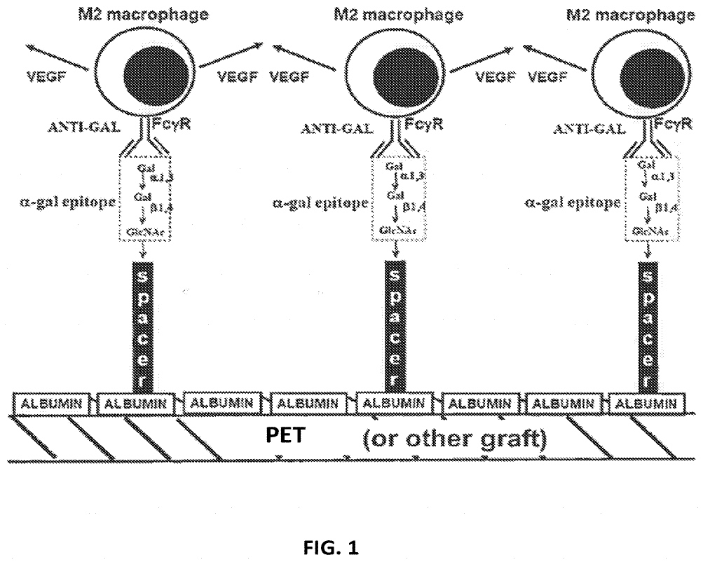

Provided herein are methods of inducing enhancement of endothelium growth (referred to as endothelialization) on the surface of medical devices, such as synthetic grafts, biologic grafts and devices, by attracting endothelial cells and progenitor endothelial cells to the surface of the implanted medical devices and further induction of rapid proliferation of said endothelial cells and progenitor endothelial cells on the surface. In some embodiments, the enhanced endothelialization may occur on a luminal surface of the implanted medical device. Attraction and induction of proliferation of the endothelial cells may be achieved by local continuous macrophage mediated generation of vascular endothelial growth factor (VEGF) on the surface of the implanted medical devices (Sunderkötter et al., 55: 410, 1994). Local continuous production of VEGF on the surface the implanted medical device is feasible by induction of monocytes/macrophages adhesion to the surface and induction of the adherent cells to polarize into prohealing M2 macrophages that secrete VEGF. Provided herein are methods for induction of monocytes/macrophages adhesion to surface of the medical devices. The adhering macrophages further polarize into M2 macrophages that secrete VEGF for periods of several days. The secreted VEGF further recruits endothelial cells and progenitor endothelial cell and induces their rapid proliferation for the covering of the intravascularly implanted medical devices, such as vascular and intracardial grafts and devices, with endothelial cells within several days post implantation. This process is achieved by using medical devices that present multiple α-gal epitopes on the surface of the medical device. These α-gal epitopes attract circulating natural anti-Gal antibody from the subject by binding anti-Gal antibody which further binds monocytes/macrophages and induces the activation of monocytes/macrophages into M2 macrophages that secrete VEGF (FIG. 1). Since the activated M2 macrophages adhere via the natural anti-Gal antibody to the medical devices, such as vascular grafts made from PTE and ePTFE and to vascular devices such as stents, a portion of the VEGF secreted by the activated M2 macrophages diffuses into the medical devices and subsequently leaches out from the medical devices and attracts endothelial cells and progenitor endothelial cells, as well as induces rapid proliferation of marcrophages for enhanced epithelialization of the surface of the medical devices.

The Natural Anti-Gal Antibody and α-Gal Epitopes

Anti-Gal is the most abundant natural antibody in all humans constituting ˜1% of circulating immunoglobulins (Galili et al. J Exp Med 160:1519, 1984; Galili, Immunology 140:1, 2013). Anti-Gal binds specifically to a carbohydrate antigen called the α-gal epitope with the structure Galα I-3Galβ1-4GlcNAc-R (Galili et al. J Exp Med 162:573, 1985). The anti-Gal antibody is produced throughout life in response to continuous antigenic stimulation by bacteria of the normal gastrointestinal flora (Galili et al. Infect Immun 56:1730, 1988). Anti-Gal is naturally produced also in Old World monkeys (monkeys of Asia and Africa) and in apes, however, it is absent in other mammals (Galili et al. Proc Nail Acad Sci USA 84:1369, 1987). In contrast, other mammalian species including nonprimate mammals (e.g. mice, rats, rabbits, dogs, pigs, etc.), as well as prosimians and New World monkeys (monkeys of South America), lack the anti-Gal antibody but all produce its ligand the α-gal epitope, by using a glycosylation enzyme called α1,3galactosyltransferae (α1,3GT) (Galili et al. Proc Natl Acad Sci USA 84: 1369, 1987; Galili et al. J Bioi Chem 263: 17755, 1988; Galili, Immunology 140:1, 2013).

The activity of the natural anti-Gal antibody can be harnessed for therapeutic purposes in humans by in vivo interaction with α-gal epitopes delivered into patients in several forms, including, but not limited to glycoproteins with linked natural or synthetic α-gal epitopes (called here α-gal glycoproteins), natural or synthetic glycolipids carrying α-gal epitopes (called here α-gal glycolipids) and α-gal nanoparticles presenting multiple α-gal epitopes (Galili Immunology 140:1, 2013; Galili patents U.S. Pat. Nos. 5,879,675; 6,361,775; 7,820,628; 8,084,057; 8,440,198; 8,865,178). The studies on therapeutic effects of antiGal interaction with α-gal epitopes cannot be performed in standard experimental animal models since mice, rats, guinea-pigs, rabbits and pigs, all produce α-gal epitopes on their cells by the glycosylation enzyme al, 3GT and thus, cannot produce the anti-Gal antibody (Galili et al. Proc Natl Acad Sci USA 84: 1369, 1987; Galili et al. J Bioi Chem 263: 17755, 1988). In addition to Old World monkeys, the only two nonprimate experimental animal models which are suitable for anti-Gal studies are α1,3GT knockout mice (GT-KO mice) produced in the mid-1990s (Thall et al. J. Biol Chem 270:21437, 1995; Tearle et al. Transplantation 61:13, 1996) and al, 3GT knockout pigs (GT-KO pigs) produced in the last decade (Lai et al. Science 295:1089, 2002; Phelps et al. Science 299:41, 2003). These two knockout animal models lack α-gal epitopes and can produce anti-Gal. Old World monkeys, which naturally produce the anti-Gal antibody can serve as animal models, as well. By using the experimental animal model of GT-KO mice it was shown that injection of α-gal glycolipids into tumor lesions induces the regression of these lesions and conversion of the injected tumor lesions into vaccines against autologous tumor antigens, thereby inducing a protective immune response against distant metastases (Galili et al. J Immunol 178: 4676, 2007; Abdel-motal et al. Cancer, Immunol. Immunopath 58: 1545, 2009). Administration of α-gal nanoparticles results in binding of the anti-Gal antibody to α-gal epitopes on these nanoparticles and the subsequent recruitment and activation of macrophages to secrete VEGF and other pro-healing cytokines (Wigglesworth et al J. Immunol 186: 4422, 2011; Galili, Adv Wound Care 6: 81, 2017).

Interaction of natural anti-Gal antibody with α-gal epitopes results in macrophage recruitment and activation into cells secreting VEGF

The in vivo ability of anti-Gal interacting with α-gal epitopes to induce macrophage recruitment and activation for VEGF secretion was demonstrated in anti-Gal producing GT-KO mice that were injected with α-gal nanoparticles. α-gal nanoparticles are submicroscopic α-gal liposomes composed of glycolipids with multiple α-gal epitopes (α-gal glycolipids), phospholipids and cholesterol (Wigglesworth et al J Immunol 186: 4422, 2011). Since α-gal glycolipids comprise most of the glycolipids in rabbit red blood cell (RBC) membranes and since these cell membranes are the richest source of α-gal glycolipids in mammals (Galili et al. Proc Natl Acad Sci USA 84: 1369, 1987; Egge et al. J Biol Chem 260: 4927, 1985, Galili et al. J Immunol 178: 4676, 2007), rabbit RBC membranes served as a natural source for preparation of α-gal nanoparticles (Wigglesworth et al J Immunol 186: 4422, 2011). For this purpose, glycolipids, phospholipids and cholesterol are extracted from rabbit RBC membranes in a mixture of chloroform and methanol (Galili et al. 178: 4676, 2007). The dried extract is sonicated in saline to generate liposomes (size of 0.1-100 μm) comprised of α-gal glycolipids, phospholipids and cholesterol and which present multiple α-gal epitopes of the glycolipids in the extract. These liposomes (referred to as α-gal liposomes) are further sonicated using a sonication probe into submicroscopic particles called α-gal nanoparticles. The multiple α-gal epitopes (10¹⁵ epitopes/mg nanoparticles) on the α-gal nanoparticles readily bind the anti-Gal antibody. (Galili, Adv Wound Care 6: 81, 2017).

Binding of the natural anti-Gal antibody to α-gal nanoparticles administered intradermal into anti-Gal producing GT-KO mice activates the complement system (Galili et al. BURNS 36: 239, 2010; Wigglesworth et al J. Immunol 186: 4422, 2011). The chemotactic factors C5a and C3a generated as complement cleavage peptides induce rapid recruitment of macrophages to the site of applied α-gal nanoparticles. It was further contemplated that the recruited macrophages interact via their Fcγ receptors (FcγR) with the Fc portion of anti-Gal coating the α-gal nanoparticles and that this Fc/FcγR interaction of anti-Gal opsonizing the α-gal nanoparticles with the macrophages, activates these cells into “pro-healing” macrophages that secrete a wide range of cytokines and growth factors, including VEGF. These cytokines induce accelerated healing of wounds treated with α-gal nanoparticles (Galili et al. BURNS 36: 239, 2010; Wigglesworth et al J Immunol 186: 4422, 2011; Galili, Adv Wound Care 6: 81, 2011)

The chemotactic effect of these complement cleavage peptides on macrophage recruitment could be demonstrated by histological analysis of the α-gal nanoparticles injection site. Within 24 h post intradermal injection of α-gal nanoparticles into GT-KO mice, multiple mononuclear cells were observed migrating to the injection site (Wigglesworth et al J Immunol 186: 4422, 2011). Almost all migrating cells were macrophages, as implied from the immunostaining of the cells with the macrophage specific antibody F4/80. The number of macrophages continued to increase for the next 6 days. The macrophages were characterized by increased size and ample cytoplasm suggested activation of the cells. These cells could be found in large numbers at the injection site, even on Day 14. However, by Day 21 post injection of the α-gal nanoparticles, all macrophages disappeared from the injection site. The α-gal nanoparticles induced recruitment of macrophages required activation of the complement system. This was demonstrated by the observation that inactivation of the complement system by injection of α-gal nanoparticles mixed with cobra venom factor (a complement activation inhibitor), result in no migration of macrophages to the injection site of GT-KO mice (Wigglesworth et al J Immunol 186: 4422, 2011).

The identity of cells recruited by α-gal nanoparticles, as macrophages, was further confirmed by subcutaneous implantation of biologically inert sponge discs (made of polyvinyl alcohol—PVA, 10 mm diameter, 3 mm thickness) that contained 10 mg α-gal nanoparticles. Most of the cells (>90%) retrieved from the PVA sponge discs explanted after 6-9 days were shown by immunostaining and analysis by flow cytometry that the cells were stained positively with antibodies specific to the macrophage markers CD11b and CD14 and not with antibodies to other cell populations, including B cells and T cells (Galili et al., BURNS 36: 239, 2010). Additional antibody staining indicated that most of the recruited macrophages are M2 macrophages as they are positive for IL10 and arginase staining and negative for IL12 staining.

The binding of anti-Gal opsonized α-gal nanoparticles to macrophages via Fc/FcγR interaction has been shown by scanning electron microscopy. Anti-Gal coated α-gal nanoparticles were incubated in vitro with macrophages grown from monocytes of GT-KO pigs. Within 2 h of incubation, the α-gal nanoparticles bound extensively to macrophages covering much of their surface. In addition to binding of α-gal nanoparticles with a size of 100-300 nm, binding of smaller nanoparticles of 10-30 nm could be demonstrated at higher magnifications. In the absence of anti-Gal, α-gal nanoparticles did not bind to macrophages.

The Fc/FcγR interaction between α-gal nanoparticles and macrophages signals activation of the recruited macrophages to produce a variety of pro-healing cytokines including VEGF. Such activation was studied with GT-KO mouse macrophages incubated for 24-48 h at 37° C., alone or with α-gal nanoparticles coated with anti-Gal or lacking the antibody. In the absence of anti-Gal, the macrophages incubated with α-gal nanoparticles secreted only background level of VEGF, as determined by ELISA measuring this cytokine. However, coincubation of the macrophages with anti-Gal coated α-gal nanoparticles for 24 h and 48 h resulted in secretion of VEGF by the activated macrophages, at levels that were significantly higher than the background levels.

The biological effects of this extensive secretion of VEGF by macrophages binding anti-Gal coated α-gal nanoparticles via Fc/FcγR interaction has been shown. Wounds in GT-KO pigs treated with α-gal nanoparticles for 13 days were subjected to histological evaluation and compared with saline treated wounds in the same pig. There are many more blood vessels in the granulation tissue of α-gal nanoparticles treated wounds than in saline treated wounds. The observed increase in vascularization of the wounds treated with α-gal nanoparticles is likely to be associated with elevated concentration of VEGF produced by macrophages within the granulation tissue of wounds treated with α-gal nanoparticles (Gallli, Adv Wound Care 6: 81, 2017).

Interaction of natural anti-Gal antibody with α-gal epitopes on implanted medical devices enhances endothelialization of the coated surface of medical devices

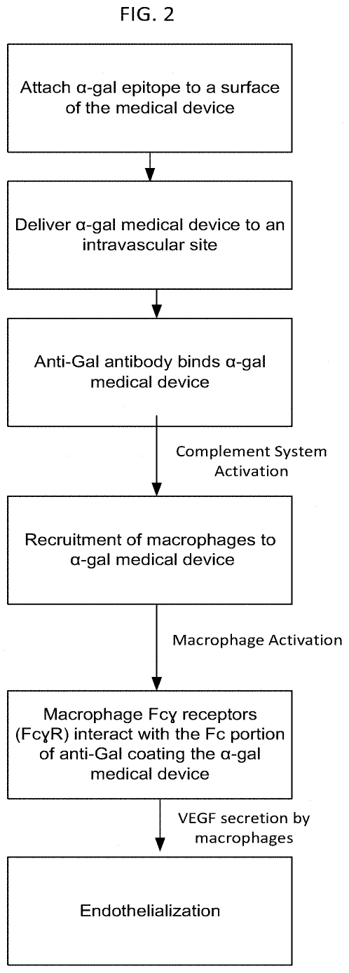

In vivo interaction of anti-Gal with α-gal epitopes coating the surface of medical devices, such as but not limited to PET and ePTFE grafts and of devices such as, but not limited to stents will enhance the endothelialization of the implanted medical devices. A PET graft coated with α-gal epitopes schematically illustrated in FIG. 1 serves as a non-limiting example for induction of such an endothelialization process. Other graft materials may also be used. Vascular grafting of PET coated with α-gal epitopes (referred to as α-gal PET) results in a series of processes occurring on the graft, as follows: 1. The natural anti-Gal antibody within the reperfusing blood binds to the α-gal epitopes coating the PET graft. 2. Monocytes/macrophages within the blood bind to the coated surface of the α-gal PET graft as a result of the interaction between the Fc portion of anti-Gal bound to the α-gal epitopes on the PET graft and Fcγ receptors (FcγR) on the monocytes/macrophages. 3. The adherent monocytes/macrophages are activated by the Fc/FcγR interaction to differentiate and polarize into M2 macrophages. 4. The activated M2 macrophages secrete VEGF. 5. The VEGF secreted through the adherent cell membranes of the macrophages attached to the PET graft penetrates the synthetic graft into the spaces between the PET fibres. 6. The secreted VEGF leaches continuously from the PET surface and generates a high local concentration of VEGF which induces enhanced division of endothelial cells ingrowths from the neighboring intact blood vessel region to cover the surface of the α-gal PET graft. In addition, the VEGF released from the α-gal PET graft induces attachment of progenitor endothelial cells to the α-gal PET surface and rapid proliferation of these progenitor endothelial cells to cover the surface of the α-gal PET graft. Ultimately, both mechanisms of proliferation and ingrowth of endothelial cells on the α-gal PET graft and the proliferation of progenitor endothelial cells differentiating into mature endothelial cells result in enhanced endothelialization of the α-gal PET graft. The process is illustrated in FIG. 2.

In some embodiments, vascular synthetic grafts coated with α-gal epitopes, such as, but not limited to, α-gal PET and ePTFE coated with α-gal epitopes (called here α-gal ePTFE) may be used as synthetic graft or “patches” in vascular surgery as grafts that undergo enhanced endothelialization, thereby decreasing the risk of post surgery thrombosis.

In other embodiments, the synthetic grafts coated with α-gal epitopes such as, but not limited to, α-gal PET and α-gal ePTFE may be used as synthetic grafts for intracardial procedures such as, but not limited to left atrial appendage closure in patients with atrial fibrillation, thereby decreasing the risk of blood clot formation in the heart.

In yet other embodiments, vascular devices such as, but not limited to, stents coated with α-gal epitopes, may be used in vascular surgery and cardiac surgery as devices that undergo enhanced endothelialization by a mechanism described above in this section and illustrated in FIG. 1. In this embodiment, the PET material is replaced by the material of the device.

Coating of Medical Devices with α-Gal Epitopes

The enhancement of endothelialization on medical devices coated with α-gal epitopes, described herein, requires the attachment of α-gal epitopes to the medical devices. Several methods for coating of medical devices with α-gal epitopes may be used, including, but not limited to: 1. Coating by a mixture of albumin and molecules of synthetic α-gal epitopes linked to albumin (referred to as α-gal albumin), all cross-linked by glutaraldehyde or other cross-linkers known to those skilled in the art, 2. Coating by a mixture of albumin and α-gal albumin which are denatured on the medical devices by heat. 3. Direct linking of α-gal epitopes via a linker to atoms of the medical devices. 4. Coating of medical devices by natural glycoproteins carrying α-gal epitopes.

1. Coating by Albumin Mixed with α-Gal Albumin and Cross-Linking to Medical Devices

Methods for coating medical devices with α-gal epitopes may use a mixture of albumin and α-gal albumin. Albumin and α-gal albumin may be cross-linked by glutaraldehyde or any other cross-linker known to those skilled in the art, as illustrated in FIG. 1. By way of non-limiting example, additional cross-linkers include formaldehyde, alcohols, succinaldehyde, octanedialdehyde and glyoxol. The example in FIG. 1 includes PET as a synthetic vascular graft, however, the described method is applicable to any kind of medical device. Albumin and α-gal albumin are mixed at any ratio between 100,000:1 to 1:1 (mol/mol) and preferably 1000:1 or 100:1. The mixed solution of albumin and α-gal albumin is referred to as “albumin/α-gal albumin”). The PET graft is soaked in albumin/α-gal albumin solution, then kept in a closed space over a glutaraldehyde solution at any glutaraldehyde concentration between 1% and 70% and preferably 50%, for 24-48 hours at room temperature. Glutaraldehyde is a molecule which has two arms, each forming a covalent link with amino groups on side chains of lysine or at the amino ends of protein molecules. The glutaraldehyde in the vapors cross-link between the multiple lysines and amino ends of protein molecules to form a coat of albumin/α-gal albumin on the PET synthetic graft. Alternatively, the PET graft soaked with albumin/α-gal albumin may be sprayed with glutaraldehyde aerosol. Subsequently, the processed α-gal PET graft is immersed for 1-24 hours in a solution of 0.1 M glycine in order to block any free aldehyde groups of glutaraldehyde molecules that are attached to the protein molecules only with one of the two reactive arms. The albumin used for this purpose may be of any mammalian source, and in some embodiments human albumin and human α-gal albumin are used in order to minimize any antigenicity of the albumin/α-gal albumin coat on the medical devices. Linking of α-gal epitopes via a spacer to albumin is performed by established methods known to those skilled in the art. The coating may be performed by a solution of only α-gal albumin. However, the mixed solution of albumin/α-gal albumin may improve the retention of cross-linked α-gal albumin on the medical device. It should be stressed that a number of studies demonstrated coating of PET only with albumin cross-linked by cross-linkers such as, but not limited to glutaraldehyde (Waroequier-Clérout et al., Biomaterials 8: 118, 1987), carbodiimide (Warocquier-Clérout et al., Biomaterials 8: 118, 1987) and sulphosuccinimidyl 4-(N-maleimidomethyl) cyclohexane-I-carboxylate (Phaneuf et al., Biomaterials 18:755, 1997) to generate “albuminated PET”. However, to our knowledge, no coating of PET, ePTFE and stents by α-gal epitopes has been described in the scientific literature.

2. Coating Medical Devices with Albumin/α-Gal Albumin Denatured by Heat

A medical device will be soaked in albumin/α-gal albumin mixture and undergo steam-autoclave at various temperatures, for example 131° C. and various pressures, for example 30 psi, for various periods of time, for example 3 minutes and will undergo coating by the mixture of albumin/α-gal albumin. (see Rumisek et al., J Vasc Surg 4: 136, 1986). In some embodiments, the cross-linking method described above will be combined with the heat method for coating the medical device with the albumin/α-gal albumin mixture.

3. Direct Linking of α-Gal Epitopes to the Medical Device

A molecule comprising of the α-gal epitope and a linker covalently attached to the α-gal epitope may be further linked via the free arm of the linker directly in a covalent bond to atoms of molecules of the medical device. Example linkers include, but are not limited to an amide linker, a disulfide linker, a thioether linker, a hydrazone linker, a hydrazide linker, an imine or oxime linker, an urea or thiourea linker, an amidine linker, an amine linker, and a sulfonamide linker. An example for such a structure is illustrated in FIG. 1 in which α-gal epitopes are linked to one of the arms of a linker. The other arm of the linker is linked to albumin. Similarly, synthetic α-gal epitopes linked to various linkers may be directly linked via a second arm of a linker to the medical by linking methods known to those skilled in the art.

For detailed descriptions of available conjugation methods, see Hermanson G T “Bioconjugate Techniques”, 2nd Edition Published by Academic Press, Inc., 2008.

4. Coating by Natural Glycoproteins Carrying α-Gal Epitopes

Several mammalian glycoproteins were found to carry multiple α-gal epitopes. Non-limiting examples are mouse laminin with −50 α-gal epitopes/molecule and bovine thyroglobulin with 12 α-gal epitopes/molecule (Arumugham et al., Biochim. Biophys. Acta 883: 112, 1986; Spiro and Bhoyroo, J Biol Chem. 259, 9858, 1984; Thall and Galili Biochemistry 29: 3959, 1990). It is contemplated that natural glycoproteins carrying α-gal epitopes may be purified and used for coating of medical devices by any of the three coating methods described above.

Purification of glycoproteins from natural sources may be done by selectively capturing the glycan component utilizing affinity chromatography. The most common affinity matrices are m-aminophenylboronic acid-agarose for nonspecific binding of saccharides, and immobilized lectins for binding specific carbohydrates. Other purification methods known in the art may also be used. In some embodiments purified laminin and thyroglobulin may also be purchased from commercial sources.

As described herein, α-gal epitopes may be conjugated to a suitable vehicle. Suitable vehicles include, but are not limited to polyethylene glycol (PEG), polyoxyethylene glycol, or polypropylene glycol, albumin, transferrin, and the like. In some embodiments, a derivative vehicle comprises one or more of monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers. These and other suitable vehicles are known in the art. Such conjugated α-gal epitopes may be in monomeric, dimeric, tetrameric, or other form. In one embodiment, one or more α-gal epitope is bonded at one or more specific position, for example at the amino terminus, of a binding agent.

Preparation of α-Gal Epitopes

### Example 1

Exemplary α-gal epitopes are generated from extracts of rabbit red blood cell (RBC) membranes. These membranes are used since they contain glycolipids carrying from one to more than seven α-gal epitopes per molecule as disclosed in Eto et al., Biochem. (Tokyo) 64, 205, (1968); Stellner et al., Arch. Biochem. Biophys. 133, 464 (1973); Dabrowski et al., J. Biol. Chem. 259, 7648 (1984) and Hanfland et al., Carbohydr. Res. 178, 1 (1988), all of which are hereby incorporated by reference. However, α-gal epitopes may be produced from any natural or synthetic source of α-gal epitopes and may include the addition of phospholipids in the presence or absence of cholesterol, after processing as described herein. As a non-limiting example, rabbit RBC are used at a volume of 0.25 liter packed cells. The RBC are lysed by repeated washes with distilled water. The rabbit RBC membranes are then mixed with a solution of 600 ml chloroform and 900 ml methanol for 20 h with constant stirring to dissolve the membrane glycolipids, phospholipids and cholesterol into the extracting solution. In contrast, proteins are denatured and are precipitating within and upon the membranes. Subsequently, the mixture is filtered to remove non-solubilized fragments and denatured proteins. The extract contains the rabbit RBC phospholipids, cholesterol and glycolipids, dissolved in the organic solution of chloroform and methanol. With the exception of the glycolipid ceramide tri-hexoside (CTH) having the structure Galα1-4Galβ1-4Glc-Cer, the glycolipids extracted from rabbit RBC membranes generally have 5 to more than 25 carbohydrate units in their carbohydrate chains with one or several branches, all of which are capped with α-gal epitopes. Rabbit RBC glycolipids were also reported to have 30, 35 and even 40 carbohydrate units with α-gal epitopes on their branched carbohydrate chains as provided for in Honma et al., J. Biochem. (Tokyo) 90, 1187 (1981), incorporated in its entirety by reference. The extract containing glycolipids, phospholipids and cholesterol is subsequently dried in a rotary evaporator. The amount of dried extract is approximately 300 mg per 0.25 liter of packed rabbit RBC. In some embodiments, human RBCs may be used for purification of the α-gal epitopes according to the protocol described herein.

### Example 2

In some embodiments, human or rabbit or bovine RBC may be used as a source of α-gal epitopes. Isolation of α-gal epitopes from rabbit RBC are used by way of non-limiting example herein. Batches of 1 liter rabbit RBC were lysed in water and washed repeatedly to remove hemoglobin. For the extraction process, rabbit RBC membranes (RBC ghosts) were mixed with 1000 ml chloroform and 1000 ml methanol (1:1 chloroform:methanol) for 2 h, then 1000 ml methanol was added for overnight incubation with constant stirring (1:2 chloroform:methanol). The extract was filtered under vacuum through Whatman filter paper for removing residual RBC membranes and precipitated proteins. For use for attaching the α-gal epitopes to a medical device, the isolated α-gal epitopes will be resuspended in a solution suitable for attaching to the medical device. The α-gal epitope suspension will be sterile because all proteins were denatured and removed in the chloroform:methanol extraction process.

### Example 3

In some embodiments, α-gal glycolipids may be used, for example from human, bovine or rabbit sources. By way of non-limiting example a rabbit source of α-gal glycolipids is described herein. A rich source for α-gal glycolipids is rabbit red blood cells. The two major glycosphingolipids (GSL), i.e. glycolipids with ceramide tail, in rabbit red blood cell membranes are ceramide (Cer) trihexoside (CTH, with 3 sugars [hexoses], present also in human red blood cells) with the structure Galα1-4Galβ1-4Glc-Cer and ceramide pentahexoside (CPH, with 5 sugars [hexoses]) with α-gal epitopes as terminal part of the CPH structure of Galα1-3Galβ1-4GlcNAcβ1-3Galβ1-4Glc-Cer (Eto T, Iichikawa Y, Nishimura K, Ando S, Yamakawa T J. Biochem. (Tokyo) 1968; 64: 205-13, and Stellner K, Saito H, Hakomori S. Arch. Biochem. Biophys. 1973; 133: 464-72) (See FIG. 1 and FIG. 6A, lane 1). Immunostaining of rabbit α-gal glycolipids on thin layer chromatography (TLC) plates with human natural anti-Gal resulted in binding of the antibody to CPH but not to CTH (See FIG. 6A, lane 2). This immunostaining revealed also that glycolipids with longer carbohydrate chains with α-gal epitopes bind anti-Gal, but migrate less than CPH because of their length.

Relatively large amounts of α-gal glycolipids were extracted from one liter of rabbit red blood cells. The red cell membranes were obtained by lysing the red blood cells with hypotonic shock in water and the membranes washed to remove the hemoglobin. The glycolipids, cholesterol and phospholipids were extracted by mixing ^{˜}140 gm of the rabbit red blood cell membranes with 600 ml chloroform and 300 ml methanol (chloroform:methanol 1:2) for 2 hours, addition of 300 ml methanol for 2 hours extraction in chloroform:methanol 1:1 and addition of methanol to a total volume of 1600 ml for overnight extraction. Extraction of glycolipids is not limited to this procedure and may be achieved by chloroform and methanol, or other solvents. In the described method, all proteins were denatured and removed by filtration through Whatman paper.

Gradual addition of 400 ml pyrogen free sterile, distilled water resulted in partitioning into approximately 500 ml of a lower organic phase containing most of the chloroform and approximately 1500 ml of an upper aqueous phase containing most of the water. Methanol was present in both phases. The lower organic phase was highly lipophilic (i.e. hydrophobic) and contained the membrane phospholipids and cholesterol (both hydrophobic molecules). Most of the CTH preferentially remained in the organic phase because the hydrophobicity of the ceramide tail (See FIG. 6B). A portion of CPH was also retained in the lower organic phase (See FIG. 6B). The upper aqueous phase was hydrophilic and contained much of the CPH and α-gal glycolipids with longer carbohydrate chains, due to the hydrophilic characteristics of the carbohydrate chains with ≥7 sugar units (See FIG. 6B). Many of these long chain glycolipids were present in the bands beneath the CPH band of the aqueous phase (See FIG. 6B). These bands include, but are not limited to, glycolipids with 7, 10, 15 and even 30 carbohydrate forming carbohydrate chains that were previously shown to have terminal α-gal epitopes (Dabrowski U, Hanfland P, Egge H, Kuhn S, Dabrowski J. J Biol. Chem. 1984 25; 259: 7648-51 and Honma K, Manabe H, Tomita M, Hamada A. 1981; 90: 1187-96) and thus, they readily bind anti-Gal. (See FIG. 6A). Note that the aqueous phase was devoid of phospholipids and cholesterol (i.e. was phospholipids-free and cholesterol-free). The methanol and traces of chloroform were removed from the upper aqueous phase in a rotary evaporator, the α-gal glycolipids were concentrated in water to 30 mg/ml. The α-gal glycolipids fully dissolved in aqueous solution creating a solution of micelles. (See FIG. 2).

Extracted rabbit red blood cell α-gal glycolipids were fractionated by high pressure liquid chromatography (HPLC) and stained on TLC plates with a monoclonal anti-Gal antibody. The separation demonstrated a CPH with a 5 carbohydrate chain, as well as CHH (ceramide heptahexoside) with a 7 carbohydrate chain and a biantennary ceramide decahexoside (Cdeca) with 10 carbohydrate chains, all with terminal α-gal epitopes (See FIGS. 6C & 6D) (Buehler J, Galili U, Macher B A. Anal Biochem. 1987; 164: 521-25). The α-gal glycolipid also included molecules with more than 10 carbohydrates (See FIG. 6A, lane 2, low band; and FIG. 6B, aqueous phase lane, low band) (Dabrowski U, Hanfland P, Egge H, Kuhn S, Dabrowski J. J Biol. Chem. 1984; 259: 7648-51). These long chain glycolipids included also α-gal glycolipids with 15 carbohydrate units (i.e., for example, Cpentadeca comprising three antennae and Cdeca comprising two antennae), and longer chain α-gal glycolipids with an average of 30 carbohydrates reported to be present in these red blood cells as “mega-glycolipids” (Honma K, Manabe H, Tomita M, Hamada A. J Biochem (Tokyo). 1981; 90: 1187-96). These types of compounds have been characterized by NMR using rabbit red blood cell glycolipids (Dabrowski U, Hanfland P, Egge H, Kuhn S, Dabrowski J. J Biol. Chem. 1984; 259: 7648-51). This preparation of α-gal glycolipids from rabbit red blood cells contained approximately 2×10¹⁶ α-gal epitopes per mg of glycolipids.

The use of α-gal glycolipids in the clinical setting may require much more material than that isolated from rabbit red blood cell membranes. The logistic limitations in supplies of large amounts of rabbit red blood cells can be overcome by the use of bovine red blood cells (i.e., for example, cow) instead of rabbit red blood cells since there is no practical limit to the amounts of bovine blood which can be supplied for preparation of α-gal glycolipids. Bovine red blood cells are suitable for α-gal glycolipids extraction since they contain CPH, CHH and α-gal glycolipid with longer carbohydrate chains (Uemura K, Yuzawa M, Taketomi T. J Biochem (Tokyo). 1978; 83: 463-71, and Chien J L, Li S C, Li Y T. J Lipid Res. 1979; 20: 669-73). Immunostaining of α-gal glycolipids extracted from bovine red blood cells demonstrated the abundance of CPH, CHH and long chain glycolipids with α-gal epitopes which readily interact with anti-Gal (See FIG. 7). Because bovine red blood cell glycolipids include many gangliosides (i.e. glycolipids with terminal sialic acid groups), preparation of α-gal glycolipids from these red blood cell membranes may require an additional step of removal of the gangliosides by the use of DEAE-Sephadex, DEAE-Sepharose columns, or any other method known in the art.

Examples of Medical Devices

Any medical device suitable for implantation and enhanced endothelialization may be coated with α-gal epitopes. The examples disclosed herein are meant to be non-limiting. In some embodiments, the medical devices may be prosthetic heart valves including mechanical or biological valves. In other embodiments the medical devices may be stents, including balloon expandable or self expanding stents, bare metal or drug eluting stents, arterial stents, such as coronary, peripheral, renal arterial, carotid, aortic, intracranial and mesenteric, and venous stents. In yet other embodiments, the medical devices may include intravascular occlusion devices, including left atrial appendage, inter-atrial septal, and devices for occlusion of a ventricular septal defect, patent ductus arteriosus, peri-valvular leak, inferior vena cava filters and intravascular grafts and patches.

In some embodiments, the medical device may be made from a polyment or a metal and combinations thereof. Non-bioresorbable, or biostable polymers that may be used include, but are not limited to, polytetrafluoroethylene (PTFE) (including expanded PTFE), polyethylene terephthalate (PET), polyurethanes, silicones, and polyesters and other polymers such as, but not limited to, polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins, polycarbonates; polyoxymethylenes; polyimides; polyethers; epoxy resins, polyurethanes; rayon; and rayon-triacetate. Examples of biologically compatible metals include, but are not limited to, stainless steel, titanium, tantalum, gold, platinum, copper and the like, as well as alloys of these metals; synthetic polymeric materials; low shape memory plastic; a shape-memory plastic or alloy, such as nitinol

The above Figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

1. A method of increasing endothelialization on a surface of a medical device, the method comprising delivering the medical device to an intravascular site, the medical device having an α-gal epitope attached to the surface of the medical device.

2. The method according to claim 1, wherein the α-gal epitope is attached to the surface comprising an intraluminal surface of the medical device.

3. The method according to claim 1, wherein the intravascular site is within an artery or a vein.

4. The method according to claim 1, wherein the intravascular site is a cardiac site.

5. The method according to claim 1, wherein the medical device comprises a synthetic or biological device.

6. The method according to claim 1, wherein the medical device is selected from the group consisting of a valve, a stent, an occlusion device, a graft or a synthetic blood vessel.

7. The method according to claim 1, wherein the medical device comprises polyethylene terephthalate (PET) or polytetrafluoroethylene (PTFE) or expanded polytetrafluoroethylene (ePTFE).

8. An implant device comprising a medical device comprising an α-gal epitope attached to a surface of the medical device.

9. The implant device according to claim 8, wherein the α-gal epitope is attached to a luminal surface of the medical device.

10. A method of making an implant device, the method comprising:
attaching an α-gal epitope to a surface of a medical device.

11. The method according to claim 10, comprising attaching the α-gal epitope to a luminal surface of the medical device.

12. The method according to claim 10, comprising attaching the α-gal epitope by crosslinking the α-gal epitope to the surface of the medical device.

13. The method according to claim 10, wherein the α-gal epitope is linked to albumin and the albumin linked α-gal epitope is attached to the surface of the medical device by crosslinking.

14. The method according to claim 10, comprising attaching the α-gal epitope by denaturing the α-gal epitope onto the surface of the medical device with heat.

15. The method according to claim 10, wherein the α-gal epitope is linked to albumin and the albumin linked α-gal epitope is attached to the surface of the medical device by denaturing with heat.

16. The method according to claim 10, wherein the α-gal epitope is attached to the surface of the medical device with a linker.

17. The method according to claim 10, wherein the α-gal epitope is purified from a mammalian glycoprotein source.

18. The method according to claim 17, wherein the α-gal epitope is purified from mouse laminin or bovine thyroglobulin.
